(19) United States
(12) Patent Application Publication
(10) Pub. No.: US 2011/0020225 A1
(43) Pub. Date: Jan. 27, 2011

(54) POROUS POLYMER PARTICLES IMMOBILIZED WITH CHARGED MOLECULES AND METHOD FOR PREPARING THE SAME

(75) Inventors: Bong Hyun Chung, Daejeon (KR); Yong Taik Lim, Daejeon (KR); Jung Hyun Han, Chungcheongnam-do (KR)

Correspondence Address: MOORE & VAN ALLEN PLLC, P.O. BOX 13706, Research Triangle Park, NC, 27709, US

(73) Assignee: KOREA RESEARCH INSTITUTE OF BIOSCIENCE AND BIOTECHNOLOGY, Daejeon (KR)

(21) Appl. No.: 12/672,517

(22) PCT Filed: Aug. 5, 2008
(86) PCT No.: PCT/KR08/04540

§ 371 Date: Mar. 17, 2010

(30) Foreign Application Priority Data
Aug. 7, 2007 (KR) 10-2007-0079058

(51) Int. Cl. A61K 49/00; A61K 9/14; A61P 31/00

(52) U.S. Cl. 424/9.1; 424/499

(57) ABSTRACT

The present invention relates to porous polymer particles containing a charged molecule immobilized therein and a method for preparing the same. According to the disclosed invention, porous particles can be prepared using a biocompatible polymer and, at the same time, a charged molecule can be immobilized in the pores of the porous particles, such that various charged molecules can be loaded in the porous particles. In addition, various kinds of drugs or functional materials can be loaded into the porous particles of the present invention by electrostatic attraction and absorption or adsorption by a capillary phenomenon occurring due to porous properties.

### TECHNICAL FIELD

The present invention relates to porous polymer particles containing a charged molecule immobilized therein and a method for preparing the same.

### BACKGROUND ART

Biocompatible, biodegradable polymers are widely used in the medical field as surgical sutures, membranes for inducing tissue regeneration, protective membranes for wound healing, and drug carriers, etc. Among biodegradable polymers, particularly polylactide (PLA), polyglycolide (PGA) and lactide-glycolide copolymer (PLGA) have been much studied and are already commercialized, because they have excellent biocompatibility and are degraded in vivo into materials harmless to the human body, such as carbon dioxide and water.

Particularly, the technology of preparing porous particles in order to use the biodegradable, biocompatible polymers as drug carriers has received increasing attention. As a representative example, a method of preparing porous particles by adding a material (porogen) capable of forming pores in polymers was reported (Park, T. G. et al., Biomaterals, 27:152, 2006; Park, T. G. et al., J. Control Release, 112:167, 2006).

Meanwhile, other examples of porous particles for use as drug carriers include silica xerogel having disordered porosity in the structure thereof, and mesoporous silica having very uniform pore size and regular pore arrangement. Porous silica is biocompatible, and it is degraded in vivo into low-molecular-weight silica by the hydrolysis of the siloxane bonds, and then released to tissue around implants. Then, it is passed through blood vessels or lymph vessels and excreted via the kidneys, in urine.

To control the release rate of drugs, studies on organic-inorganic complexes of silica xerogel and P(CL/DL-LA) (Poly(ε-caprolactone-co-DL-lactide)) polymer are now in progress (International J. Pharmaceutics, 212:121, 2001).

In addition, there are several articles reported the synthesis of porous carbon materials using templates. For example, a novel technology of synthesizing macroporous carbon materials having regular pore arrangement and uniform pore size, by introducing precursors, such as carbohydrates or polymeric monomers, into colloidal crystal templates with spherical silica particles, subjecting the precursors to polymerization and carbonization processes, and then melting and removing the templates, was reported (Zajhidov A. A. et al., Science, 282:879, 1998).

Such porous particles are used as carriers or vehicles for delivering drugs, genes, proteins or the like or as cell scaffolds for cell proliferation, but the above-described prior technologies have shortcomings in that porous particles must use separate templates for forming pores and in that materials, which can be loaded in the pores of porous particles, are limited.

Accordingly, the present inventors have made many efforts to solve the above-described problems occurring in the prior art, and as a result, have found that, through the use of a double emulsion method, porous polymer particles can be prepared and, at the same time, a charged molecule can be immobilized to the inside of the porous polymer particles, and molecules having a charge opposite to that of the charged molecule can be loaded in the porous polymer particles containing the charged molecule immobilized therein, thereby completing the present invention.

### SUMMARY OF INVENTION

It is an object of the present invention to provide porous polymer particles containing a charged molecule immobilized therein and a method for preparing the same.

To achieve the above object, the present invention provides a method for preparing porous polymer particles containing a charged molecule immobilized therein, the method comprising the steps of: (a) dispersing a mixed aqueous solution of a charged molecule and a protein having affinity for the charged molecule, in an organic solution of polymer to prepare a first dispersion; (b) dispersing the first dispersion in an aqueous solution of an emulsifier to prepare a second dispersion; and (c) stirring and separating the second dispersion to remove an organic solvent used for preparing the organic polymer solution of step (a), and the emulsifier of step (b), and then collecting porous polymer particles from the stirred dispersion.

In the present invention, the polymer is preferably a biodegradable polyester polymer. The biodegradable polyester polymer is preferably selected from the group consisting of poly-L-lactic acid, poly glycol acid, poly-D-lactic acid-co-glycol acid, poly-L-lactic acid-co-glycol acid, poly-D,L-lactic acid-co-glycol acid, poly-caprolactone, poly-valerolactone, poly-hydroxy butyrate and poly-hydroxy valerate.

In the present invention, the organic solvent used for preparing the organic polymer solution is preferably one or a mixed solvent of two or more selected from the group consisting of methylene chloride, chloroform, ethyl acetate, acetaldehyde dimethyl acetal, acetone, acetonitrile, chloroform, chlorofluorocarbons, dichloromethane, dipropyl ether, diisopropyl ether, N,N-dimethylformamide, formamide, dimethyl sulfoxide, dioxane, ethyl formate, ethyl vinyl ether, methyl ethyl ketone, heptane, hexane, isopropanol, butanol, triethylamine, nitromethane, octane, pentane, tetrahydrofuran, toluene, 1,1,1-trichloroethane, 1,1,2-trichloroethylene and xylene.

In the present invention, the protein having affinity for the charged molecule is preferably selected from the group consisting of serum protein, serum albumin, lipoprotein, transferrin, and peptide complexes having a molecular weight of more than 100.

In the present invention, the charged molecule is preferably selected from the group consisting of dyes, fluorescent dyes, therapeutic agents, diagnostic reagents, antimicrobial agents, contrast agents, antibiotic agents, fluorescent molecules, and molecules targeting specific molecules. The molecule targeting specific molecules is preferably one or a combination of two or more selected from the group consisting of antibodies, polypeptides, polysaccharides, DNA, RNA, nucleic acids, lipids and carbohydrates.

In the present invention, the emulsifier is preferably selected from the group consisting of PVA, nonionic surfactants, cationic surfactants, anionic surfactants and amphoteric surfactants.

In another aspect, the present invention provides porous polymer particles, which are prepared according to said method, contain a charged molecule immobilized therein and have a particle diameter of 1-1000 μm and a pore diameter between 100 nm and 100 μm.

In still another aspect, the present invention provides a drug carrier in which a drug is bound to a charged molecule in porous polymer particles. In the drug carrier of the present invention, the binding of the drug is achieved by a method selected from the group consisting of electrostatic attraction, absorption and adsorption.

Other features and aspects of the present invention will be more apparent from the following detailed description and the appended claims.

### BRIEF DESCRIPTION OF DRAWINGS

FIG. 1 is a schematic diagram showing the inventive process for preparing porous polymer particles containing a charged molecule immobilized therein.

FIG. 2 is a SEM photograph of porous PLGA/HSA/ICG microparticles prepared according to the present invention.

FIG. 3 is a SEM photograph of porous PLGA/HSA/Ru-Dye microparticles prepared according to the present invention.

FIG. 4 is a SEM photograph of porous PLGA/HSA/PEI microparticles prepared according to the present invention.

FIG. 5 is a SEM photograph of porous PLGA/HSA/PSS particles prepared according to the present invention.

FIG. 6 is a fluorescence micrograph of porous PLGA/HSA/PEI microparticles prepared according to the present invention, which have ICG-fluorescent dye charge-coupled thereto.

FIG. 7 is a fluorescence micrograph of porous PLGA/HSA/PEI microparticle, prepared according to the present invention, which have ovalbumin-fluorescent dye charge-coupled thereto.

### DETAILED DESCRIPTION OF THE INVENTION AND PREFERRED EMBODIMENTS

The present invention is characterized in that a double emulsion method is used to prepare porous polymer particles and, at the same time, immobilize a charged molecule to the inside of the porous polymer particles, and other molecules having a charge opposite to that of the charged molecule are loaded in the porous polymer particles containing the charged molecule immobilized therein.

In the present invention, the double-emulsion method employs water-in-oil-in-water (W₁/O/W₂) emulsion. Specifically, the double-emulsion method is a method in which a water-soluble material is impregnated again into oil-phase polymer particles dispersed in an aqueous solution (Cohen, S. et al., Pharm. Res., 8: 713, 1991).

In the present invention, according to the double-emulsion method, porous polymer particles containing charged molecules immobilized therein are prepared by dispersing a mixed aqueous solution of a protein and a charged molecule in an organic solution of polymer, and then dispersing the organic polymer solution, containing the mixed aqueous solution dispersed therein, in an aqueous solution of an emulsifier.

As the polymer that is used in the present invention, a biodegradable polyester polymer is preferably used, and particularly PLGA is preferably used. PLGA is a polymer material approved by the US FDA and is advantageous in that, because it has no problem of toxicity, the direct application thereof for medical applications, such as drug delivery systems or biomaterials, is easier than the case of other polymers.

The protein that is used in the present invention has affinity for the charged molecules and functions as an emulsion stabilizer. Examples of the protein that can be used in the present invention include, but are not limited to, serum proteins, such as albumin, globulin or fibrinogen, serum albumin, lipoprotein, transferrin, and peptide complexes having a molecular weight higher than 100. Particularly, serum albumin is preferably used.

Generally, serum albumin has various functions, such as nutrition by non-covalent bonding, the control of osmotic pressure in the human body, and the delivery of calcium ions, various metal ions, low-molecular-weight substances, bilirubin, drugs and steroids. Also, due to the function of binding such endogenous and exogenous substances, serum albumin can be used for the treatment of diseases, such as chronic renal failure, liver cirrhosis and shock disorders, hypovolemia caused by blood loss or fluid loss (Gayathri, V. P., Drug Development Research, 58: 219, 2003).

As the charged molecule that is used in the present invention, any molecule may be used without limitation, as long as it is a negatively or positively charged molecule. The charged molecule is immobilized to the inner surface of the pores of the porous polymer particles prepared according to the present invention and it functions such that a molecule having a charge opposite to that of the charged molecule can be loaded in the porous polymer particles. Thus, the charged molecule allows the porous polymer particles to bind drugs and functional substances, such that the porous polymer particles can be used as vehicles for delivering said drugs and functional substances and as cell scaffolds.

The aqueous emulsifier solution that is used in the present invention is prepared by dissolving an emulsifier in triple-distilled water. In the present invention, an aqueous solution of polyvinyl alcohol (PVA) is particularly preferably used as the aqueous emulsifier solution. PVA functions as a surfactant for stabilizing polymer particles, and examples of emulsifiers, which can be used in the present invention, include, but are not limited to, in addition to PVA, polyalcohol derivatives, such as glycerin monostearate and stearate, nonionic surfactants, including sorbitan esters and polysorbates, cationic surfactants such as cetyltrimethyl ammonium bromide, anionic surfactants, such as sodium lauryl sulfate, alkyl sulfonate and alkyl aryl sulfonate, and amphoteric surfactants, such as higher alkyl amino acid, polyaminomonocarboxylic acid and lecithin.

In the present invention, when the mixed aqueous solution of protein and charged molecules is dispersed in the organic polymer solution, it is preferably dispersed in a reverse emulsion (water-in-oil emulsion). Herein, the reverse emulsion refers to a state in which an aqueous phase is dispersed in an oil phase while forming droplets. In the present invention, the mixed aqueous solution of the charged material and the protein having affinity for the charged molecule, as the aqueous phase, is dispersed in the organic polymer solution while forming droplets, thus forming pores of the resulting porous polymer particles.

In addition, when the mixed aqueous solution of the charged molecule and the protein having affinity for the charged molecule is dispersed in the organic polymer solution to form droplets, the charged molecule is uniformly dispersed in each of the droplets, such that the agglomeration of the droplets of the mixed aqueous solution dispersed in the organic polymer solution is prevented by charge repulsive force, thus forming pores of the resulting porous polymer particles.

In the present invention, when the organic polymer solution, in which the mixed aqueous solution of the charged molecule and the protein having affinity for the charged molecule is dispersed, is dispersed in an aqueous emulsifier solution, the aqueous emulsifier solution forms droplets. At this time, the porous polymer particles can be obtained by removing the organic solvent from the organic polymer solution and then solidifying the polymer.

Because the charged molecule is immobilized in the pores of the porous polymer particles prepared according to the present invention, other molecules having a charge opposite to that of the charged molecule can be easily loaded in the porous polymer particles. Particularly, the porous polymer particles containing the charged molecule immobilized therein is effective in loading medical drugs therein, and thus highly useful as drug carrier.

In order to utilize the inventive porous polymer particles, containing the charged molecule immobilized therein as drug carriers, it is preferable to bind drugs to the inside of the pores of the porous polymer particles. Herein, the binding of the drug to the inside of the pores of the porous polymer particles is achieved by electrostatic attraction, absorption or adsorption.

With respect to the binding of drugs to the porous polymer particles by electrostatic attractions, the drug is bound to the pores of the porous polymer particles by the electrostatic attraction between the charged molecule immobilized in the pores of the porous polymer particles, and the drug having a charge opposite to that of the charged molecule.

In addition, a drug can also be bound to the pores of the porous polymer particles by absorption or adsorption caused by the porosity of the porous polymer particles. As used herein, the term “absorption or adsorption caused by porosity” means that an absorption or adsorption phenomenon occurring due to the properties of pores formed in porous particles.

It is generally known that porous particles prepared using activated carbon, zeolite, metal oxide or silica have the properties of capillary absorption and capillary condensation due to their small pore sizes, and the physical adsorption of other phases (e.g., gas, liquid and solid phases) into the porous particles is increased due to a large number of pores at the interface (Olivier, J. P., Studies in Surface Science and Catalysis, 149:1, 2004; Stevik, T. K. et al., Water Research, 38:1355, 2004; Steele, W., Applied Surface Science, 196: 3, 2002).

The capillary phenomenon can also be observed in the porous polymer particles prepared according to the present invention. Due to this capillary phenomenon, liquid can be absorbed and bound to the pores of the porous polymer particles, and materials to be loaded in the pores of the porous polymer particles can be bound to the pores. Particularly, the porous polymer particles of the present invention can adsorb a large amount of substances, because they have increased specific surface area due to the pores thereof.

As described above, due to the binding ability of the porous polymer particles according to the present invention, drugs, prepared using extracts of animals, plants, microorganisms or viruses as raw materials, and drugs, prepared through chemical synthetic processes, can be loaded in the porous polymer particles, and thus the porous polymer particles can be used as drug delivery systems. Furthermore, various functional materials in addition to drugs can be loaded in the porous polymer particles, and thus the porous polymer particles can be applied in various industrial fields.

Particularly, drugs, prepared using any one selected from the group consisting of animal, plant, microbial and viral extracts, include, but are not limited to, DNA, RNA, peptides, amino acids, proteins, collagens, gelatins, fatty acids, hyaluronic acid, placenta, vitamins, monosaccharides, polysaccharides, Botox and metal compounds, and drugs prepared by chemical synthetic processes include, but are not limited to, antipsychotic drugs, antidepressants, antianxiety drugs, analgesic drugs, antimicrobial agents, sedative-hypnotics, anticonvulsant drugs, antiparkinson drugs, narcotic analgesics, nonopioid analgesics, cholinergic drugs, adrenergic drugs, antihypertensive drugs, vasodilators, local anesthetics, anti-arrhythmic drugs, cardiotonic drugs, antiallergic drugs, antiulcer drugs, prostaglandin analogs, antibiotics, antifungal drugs, anti-protozoa drugs, anthelmintics, antiviral drugs, anticancer drugs, hormone-related drugs, antidiabetic drugs, antiarteriosclerotic drugs and diuretic drugs.

According to one embodiment of the present invention, porous polymer particles containing a charged molecule immobilized therein can be prepared using PLGA as the polymer, methylene chloride as the organic solvent, human serum albumin (HAS) as the emulsion stabilizer, indocyanine green (ICG) as the charged molecule, and PVA solution as the aqueous emulsifier solution.

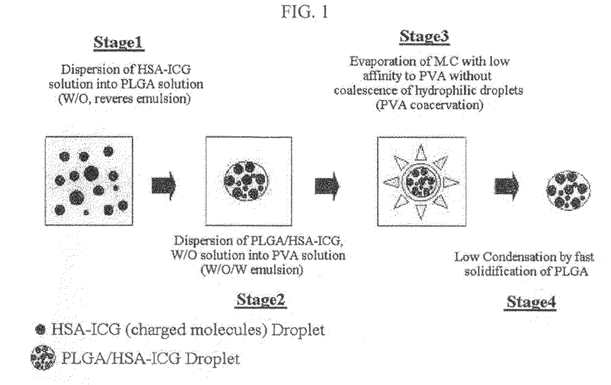

As shown in FIG. 1, in stage 1, PLGA was dissolved in a methylene chloride solvent to prepare an organic PLGA solution (O), HSA and ICG were dissolved in triple-distilled water to prepare an aqueous HSA-ICG solution (W₁), and then the aqueous HSA-ICG solution was dispersed in the organic PLGA solution to prepare a reverse emulsion (W₁/O). In stage 2, the PLGA/HSA-ICG solution dispersed as the reverse emulsion was dispersed in an aqueous PVA solution (W₂) to prepare a dispersion (W₁/O/W₂). In stage 3, the spontaneous evaporation of the methylene chloride solvent and the coacervation of PVA were observed. In stage 4, the aqueous HSA-ICG solution remained dispersed in the organic PLGA solution in the PLGA particles by the solidification of PLGA, thus exhibiting pores, and porous PLGA particles containing the HSA and ICG immobilized in the pores were collected.

As used herein, the term “coacervation” refers to a phenomenon in which hydrophilic colloids form droplets, and in the present invention, it means that the aqueous emulsifier solution forms droplets.

### EXAMPLES

Hereinafter, the present invention will be described in further detail with reference to examples. It is to be understood, however, that these examples are for illustrative purposes only and are not to be construed to limit the scope of the present invention.

### Example 1

### Preparation of porous PLGA/HAS (human serum albumin)/ICG (indocyanine Green) microparticles

100 mg of PLGA was dissolved in 2 ml of methylene chloride to prepare an organic solution of PLGA, and 15 mg of human serum albumin (HSA) and 5 mg of indocyanine green (ICG; negatively charged) were dissolved in 250 μl of triple-distilled water to prepare a mixed aqueous solution. The mixed aqueous solution was dispersed and stirred in the organic PLGA solution, and then the organic PLGA solution containing the mixed aqueous solution dispersed therein was slowly added dropwise to 30 ml of 4%-PVA solution, while it was dispersed using a homogenizer at 25000 rpm for 5 minutes. Then, the dispersed solution was stirred overnight to remove the methylene chloride solvent. Then, the remaining material was centrifuged at 8000 rpm for 10 minutes to collect porous PLGA/HSA/ICG microparticles. The supernatant was decanted, and the residue was added to distilled water, re-dispersed with ultrasonic waves, and then centrifuged again. Such decantation, dispersion and centrifugation procedures were repeated three times. Then, porous PLGA/HSA/ICG microparticles were finally collected, freeze-dried and stored at 4° C.

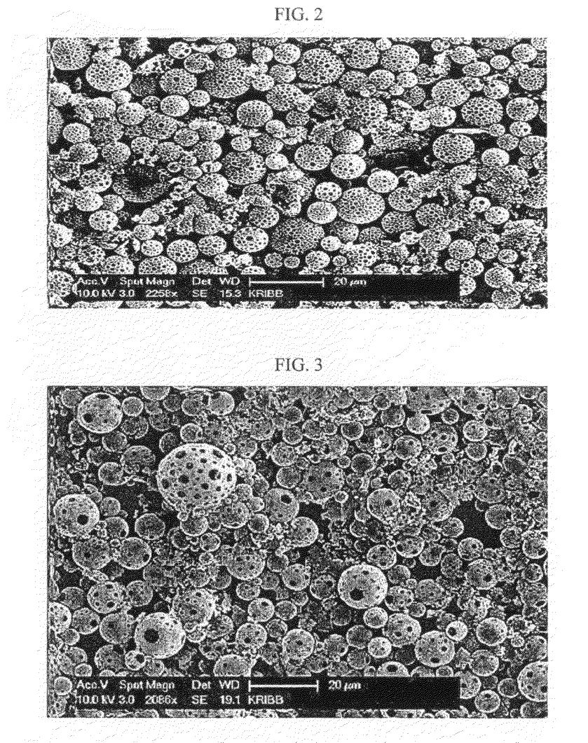

The collected porous PLGA/HSA/ICG microparticles were observed with a scanning electron microscope (SEM) and, as a result, it was found that the microparticles had a particle diameter of 1-50 μm and a pore diameter between 100 nm and 2 μm (FIG. 2).

### Example 2

### Preparation of porous PLGA/HSA/Ru-Dye[tris(2,2′-bipyridyl)dichloro-ruthenium(II) DYES] microparticles

100 mg of PLGA was dissolved in 2 ml of methylene chloride to prepare an organic solution of PLGA, and 15 mg of human serum albumin (HSA) and 5 mg of Ru-Dye (positively charged) were sequentially dissolved in 250 μl of triple-distilled water to prepare a mixed aqueous solution. The mixed aqueous solution was dispersed and stirred in the organic PLGA solution, and then the organic PLGA solution containing the mixed aqueous solution dispersed therein was slowly added dropwise to 30 ml of 4%-PVA solution, while it was dispersed using a homogenizer at 25000 rpm for 5 minutes. Then, the dispersed solution was stirred overnight to remove the methylene chloride solvent. Then, the remaining material was centrifuged at 8000 rpm for 10 minutes to collect porous PLGA/HSA/Ru-Dye microparticles. The supernatant was decanted, and the residue was added to distilled water and re-dispersed with ultrasonic waves, and then centrifuged again. Such decantation, dispersion and centrifugation procedures were repeated three times. Then, the porous PLGA/HSA/Ru-Dye microparticles were finally collected, freeze-dried and stored at 4° C.

The finally collected porous PLGA/HSA/Ru-Dye microparticles were observed with a SEM and, as a result, it was found that they had a particle diameter of 1-50 μm and a pore diameter between 100 nm and 5 μm (FIG. 3).

### Example 3

### Preparation of PLGA/HSA/PEI(polyethyleneimine) microparticles

100 mg of PLGA was dissolved in 2 ml of methylene chloride to prepare an organic solution of PLGA, and 15 mg of human serum albumin (HSA) and 5 mg of polyethyleneimine (PEI; positively charged) were sequentially dissolved in 250 μl of triple-distilled water to prepare a mixed aqueous solution. The mixed aqueous solution was dispersed and stirred in the organic PLGA solution, and then the organic PLGA solution containing the organic PLGA solution dispersed therein was slowly added dropwise to 30 ml of 4%-PVA solution, while it was dispersed using a homogenizer at 25000 rpm for 5 minutes. Then, the dispersed solution was stirred overnight to remove the methylene chloride solution. Then, the remaining material was centrifuged at 8000 rpm for 10 minutes to collect porous PLGA/HSA/PEI microparticles. The supernatant was decanted, and the residue was added to distilled water, re-dispersed with ultrasonic waves and centrifuged again. Such decantation, dispersion and centrifugation procedures were repeated three times. Then, the porous PLGA/HSA/PEI microparticles were finally collected, freeze-dried and stored at 4° C.

The finally collected porous PLGA/HSA/PEI microparticles were observed with a SEM and, as a result, it was found that they had a particle diameter of 1-50 μm and a pore diameter between 100 nm and 10 μm (FIG. 4).

### Example 4

### Preparation of porous PLGA/HSA/PSS[poly(sodium 4-styrenesulfonate)] microparticles

100 mg of PLGA was dissolved in 2 ml of methylene chloride to prepare an organic solution of PLGA, and 15 mg of human serum albumin (HSA) and 5 mg of poly(sodium 4-styrenesulfonate) (PSS; positively charged) were sequentially dissolved in 250 μl of triple-distilled water to prepare a mixed aqueous solution. The mixed aqueous solution was dispersed and stirred in the organic PLGA solution, and then the organic PLGA solution containing the mixed aqueous solution dispersed therein was slowly added dropwise to 30 ml of 4%-PVA solution, while it was dispersed using a homogenizer at 25000 rpm for 5 minutes. Then, the dispersed solution was stirred overnight to remove the methylene chloride solvent. Then, the remaining material was centrifuged at 8000 rpm for 10 minutes to collect porous PLGA/HSA/PSS microparticles. The supernatant was decanted, and the residue was added to distilled water, re-dispersed with ultrasonic waves and then centrifuged again. Such decantation, dispersion and centrifugation procedures were repeated three times. Then, the porous PLGA/HSA/PSS microparticles were finally collected, freeze-dried and stored at 4° C.

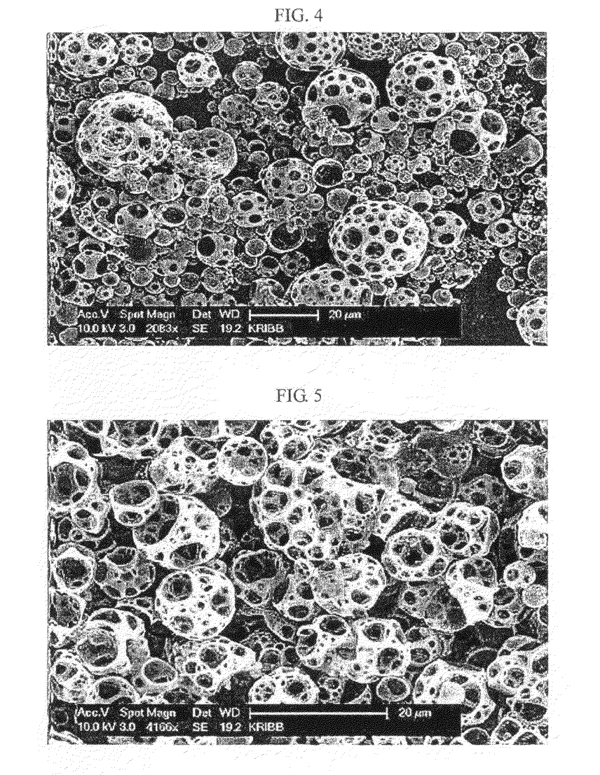

The finally collected porous PLGA/HSA/PSS microparticles were observed with a SEM and, as a result, it was found that they had a particle diameter of 1-50/cm and a pore diameter ranging from 100 nm to 10 μm (FIG. 5).

### Example 5

### Experiment of charge coupling of ICG fluorescent dye to porous PLGA/HSA/PEI (polyethyleneimine) microparticles

The porous PLGA/HSA/PEI microparticles prepared in Example 3, which comprises the positively charged molecule immobilized in the pores thereof, were added to PBS solution (pH 7.4) to prepare a solution having a concentration of about 3 mg microparticles/ml PBS. 5 mg indocyanine green (ICG) having a weak negative charge was added to 1 ml of the solution, and then stirred for 20 minutes to prepare a mixed solution. The mixed solution was centrifuged at 10000 rpm for about 5 minutes and re-dispersed in PBS solution. Such centrifugation and dispersion procedures were repeated three times, and then porous PLGA/HSA/PEI microparticles having ICG specifically charge-coupled thereto were collected from the centrifuged material.

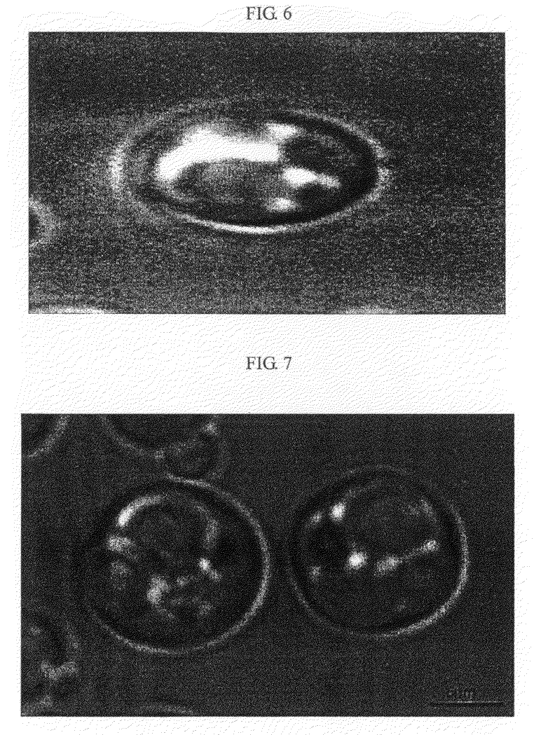

The collected porous PLGA/HSA/PEI microparticles containing ICG charge-coupled thereto were observed with a fluorescent microscope and, as a result, it was seen that ICG was charge-coupled specifically to the inside of the pores of the microparticles (FIG. 6).

### Example 6

### Experiment of charge coupling of ovalbumin-fluorescent dye to porous PLGA/HSA/PEI (polyethyleneimine) microparticles

The PLGA/HSA/PEI microparticles prepared in Example 3, which comprises the positively charged molecule immobilized in the pores thereof, were added to PBS solution (pH 7.4) to prepare a solution having a concentration of about 3 mg microparticles/ml PBS. Then, 5 mg of ovalbumin-fluorescent dye (45 kDa, pI=4.6) having a negative charge at pH 7.4 was added to 1 ml of the solution, and then stirred for 20 minutes to prepare a mixed solution. The mixed solution was centrifuged at 1000 rpm for about 5 minutes and re-dispersed in PBS solution. Such centrifugation and dispersion procedures were repeated three times. Then, porous PLGA/HSA/PEI microparticles having ovalbumin-fluorescent dye specifically charge-coupled thereto were collected (FIG. 7).

### INDUSTRIAL APPLICABILITY

As described above, according to the present invention, porous particles are prepared using a biocompatible polymer and, at the same time, a charged molecule can be immobilized in the pores of the porous particles, such that various charged molecules can be loaded in the porous particles. In addition, various kinds of drugs or functional materials can be loaded into the porous particles of the present invention by electrostatic attraction and absorption or adsorption by a capillary phenomenon occurring due to porous properties. Furthermore, the porous particles according to the present invention can be applied to columns or membranes for separation and can also be used as cell scaffolds in the tissue engineering field.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A method for preparing porous polymer particles containing a charged molecule immobilized therein, the method comprising the steps of:
(a) dispersing a mixed aqueous solution of a charged molecule and a protein having affinity for the charged molecule, in an organic solution of polymer to prepare a first dispersion;
(b) dispersing the first dispersion in an aqueous solution of an emulsifier to prepare a second dispersion; and
(c) stirring and separating the second dispersion to remove an organic solvent used for preparing the organic polymer solution of step (a), and the emulsifier of step (b), and then collecting porous polymer particles from the stirred dispersion.

2. The method for preparing porous polymer particles containing a charged molecule immobilized therein according to claim 1, wherein the polymer is a biodegradable polyester polymer.

3. The method for preparing porous polymer particles containing a charged molecule immobilized therein according to claim 2, wherein the biodegradable polyester polymer is selected from the group consisting of poly-L-lactic acid, poly glycol acid, poly-D-lactic acid-co-glycol acid, poly-L-lactic acid-co-glycol acid, poly-D,L-lactic acid-co-glycol acid, poly-caprolactone, poly-valerolactone, poly-hydroxy butyrate and poly-hydroxy valerate.

4. The method for preparing porous polymer particles containing a charged molecule immobilized therein according to claim 1, wherein the organic solvent used for preparing the organic polymer solution is one or a mixed solvent of two or more selected from the group consisting of methylene chloride, chloroform, ethyl acetate, acetaldehyde dimethyl acetal, acetone, acetonitrile, chloroform, chlorofluorocarbons, dichloromethane, dipropyl ether, diisopropyl ether, N,N-dimethylformamide, formamide, dimethyl sulfoxide, dioxane, ethyl formate, ethyl vinyl ether, methyl ethyl ketone, heptane, hexane, isopropanol, butanol, triethylamine, nitromethane, octane, pentane, tetrahydrofuran, toluene, 1,1,1-trichloroethane, 1,1,2-trichloroethylene and xylene.

5. The method for preparing porous polymer particles containing a charged molecule immobilized therein according to claim 1, wherein the protein having affinity for the charged molecule is selected from the group consisting of serum protein, serum albumin, lipoprotein, transferrin, and peptide complexes having a molecular weight of more than 100.

6. The method for preparing porous polymer particles containing a charged molecule immobilized therein according to claim 1, wherein the charged molecule is selected from the group consisting of dyes, fluorescent dyes, therapeutic agents, diagnostic reagents, antimicrobial agents, contrast agents, antibiotic agents, fluorescent molecules, and molecules targeting specific molecules.

7. The method for preparing porous polymer particles containing a charged molecule immobilized therein according to claim 6, wherein the molecule targeting specific molecules is one or a combination of two or more selected from the group consisting of antibodies, polypeptides, polysaccharides, DNA, RNA, nucleic acids, lipids and carbohydrates.

8. The method for preparing porous polymer particles containing a charged molecule immobilized therein according to claim 1, wherein the emulsifier is selected from the group consisting of PVA, nonionic surfactants, cationic surfactants, anionic surfactants and amphoteric surfactants.

9. Porous polymer particles prepared by the method of claim 1, which contain a charged molecule immobilized therein and have a particle diameter of 1-1000 μm and a pore diameter between 100 nm and 100 μm.

10. A drug carrier in which a drug is bound to a charged molecule immobilized in porous polymer particles of claim 9.

11. The drug carrier according to claim 10, wherein the binding of the drug is achieved by a method selected from the group consisting of electrostatic attraction, absorption and adsorption.
